(19) United States
(12) Patent Application Publication
(10) Pub. No.: US 2022/0395767 A1
(43) Pub. Date: Dec. 15, 2022

(54) ENHANCING LCMS ANALYTE SIGNALS

(71) Applicant: PerkinElmer Health Sciences, Inc., Waltham, MA (US)

(72) Inventor: Avinash Dalmia, Hamden, CT (US)

(73) Assignee: PerkinElmer Health Sciences, Inc., Waltham, MA (US)

(21) Appl. No.: 17/348,349

(22) Filed: Jun. 15, 2021

(51) Int. Cl. B01D 15/32; B01J 20/281; G01N 30/72

(52) U.S. Cl. CPC B01D 15/325; G01N 30/48; G01N 30/7233; G01N 2030/488

(57) ABSTRACT

This disclosure provides liquid chromatography tandem mass spectrometer (LC-MS/MS) methods and systems for detecting low levels of pesticides and mycotoxins in a test sample. In the disclosed methods and systems, oxalic acid is added to a mobile phase composition of a reverse phase chromatographic separation column. This addition improves the signal for certain pesticides and mycotoxins by a factor of from 1.5 to 9, improving their detection limits in a variety of test samples.

### TECHNICAL FIELD

This disclosure relates generally to systems and methods for detecting and/or quantifying pesticides and mycotoxins using mass spectrometry.

### BACKGROUND

Liquid chromatography tandem mass spectrometer (LC-MS/MS) methods can be used to detect pesticides and mycotoxins in a variety of test samples, including in cannabis and food matrices. It is common practice when using an electrospray ionization source (ESI) in such methods to add certain volatile chemicals or additives to the mobile phase or to introduce them post-column, prior to the ESI interface, to influence analyte ionization and improve the analyte signal.

The most commonly used additives are volatile acids (formic, acetic, and trifluoroacetic acid) and salts (ammonium formate and ammonium acetate). The acidic additives are used to enhance ionization or signal of analytes in positive ion mode by protonating them. The neutral salts are added to the mobile phase either to improve ionization efficiency of molecules that have low proton affinity in positive ion mode, by forming ammonia adducts or form ions in negative ion mode by deprotonation or adduct formation.

### SUMMARY OF THE INVENTION

The ionization efficiency of certain pesticides and mycotoxins is quite low with the use of the most commonly used additives, making it difficult to measure very low levels of these pesticides and mycotoxins in cannabis, food, water, and other matrices. Thus it is an object of the disclosure to provide improved systems and methods for detecting pesticides and/or mycotoxins in test samples to comply with rigorous and stringent testing regulations in various U.S. states and countries, such as, but not limited to, Florida, California, and Canada.

In accordance with some embodiments, the present disclosure includes a reverse phase chromatographic separation column comprising a mobile phase composition, wherein the mobile phase composition comprises a first mobile phase, a second mobile phase, and oxalic acid.

In at least some embodiments, the first mobile phase comprises water, ammonium formate, and formic acid. In some embodiments, the second mobile phase comprises methanol, ammonium formate, and formic acid.

The first mobile phase may comprise a first concentration of oxalic acid of from 10 μM to 1000 μM. In at least some embodiments, the first mobile phase and the second mobile phase independently comprise a concentration of oxalic acid of from 0 μM to 1000 μM, for example, 50 μM to 500 μM, provided that at least one of the first and second mobile phases comprises a concentration of oxalic acid greater than 0 μM. In at least some embodiments, the first mobile phase comprises 83 μM oxalic acid and/or the second mobile phase comprises 166 oxalic acid. In at least some embodiments, the first mobile phase and the second mobile phase each comprise 2 mM ammonium formate and 0.1% formic acid.

The mobile phase composition may comprise a test sample, for example, a botanical test sample, an environmental sample, a clinical sample, and/or an extract of marijuana or hemp product (e.g., flowers, concentrates, edibles, topicals, and smokables. In at least some embodiments, the test sample comprises a pesticide, such as one or more pesticides selected from the group consisting of abamectin, acequinocyl, captan, cyfluthrin, cypermethrin, daminozide, fenhexamid, flunicamide, parallethrin, permethrin, and pyrethrin I. In some embodiments, the test sample may comprise a mycotoxin, such as ochratoxin A.

In at least some embodiments, the disclosure includes a reverse phase chromatographic separation system, comprising a reverse phase chromatographic separation column, a first mobile phase comprising oxalic acid, and a second mobile phase comprising oxalic acid. In some such embodiments, the first mobile phase may comprise water, ammonium formate, and formic acid and/or the second mobile phase may comprise methanol, ammonium formate, and formic acid. In at least some embodiments, the first mobile phase comprises a first concentration of oxalic acid and/or the second mobile phase comprises a second concentration of oxalic acid. In at least some embodiments, the first and second concentrations of oxalic acid independently are from 0 μM to 1000 μM, provided that at least one of the first and second mobile phases comprises a concentration of oxalic acid greater than 0 μM. For example, the first mobile phase may comprise 83 μM oxalic acid and/or the second mobile phase may comprise 166 μM oxalic acid. In at least some embodiments, the first mobile phase and the second mobile phase each comprise 2 mM ammonium formate and 0.1% formic acid.

In at least some embodiments, the present disclosure includes liquid chromatography tandem mass spectrometer (LC-MS/MS) systems, for example, comprising a reverse phase chromatographic separation column as described above and a triple quadrupole mass spectrometer. In at least some such embodiments, the triple quadrupole mass spectrometer is configured to detect an MRM transition selected from the group consisting of 890.50/305.10 (abamectin), 402.20/343.10 (acequinocyl), 316.90/263.90 (captan), 451.10/191.00 (cyfluthrin), 433.10/191.00 (cypermethrin), 161/10/143.00 (daminozide), 302.10/97.00 (fenhexamid), 230.10/203.00 (flunicamide), 404.10/239.00 (ochratoxin A), 301.20/133.00 (parallethrin), 408.10/183.00 (permethrin), and 329.20/133.00 (pyrethrin I).

In at least some embodiments, the present disclosure includes methods for detecting a pesticide in a test sample. In some such embodiments, the method may include (1) processing a test sample using the reverse phase chromatographic separation column to provide a liquid chromatography (LC) column eluant; and (2) analyzing the LC column eluant for the presence of the pesticide or the mycotoxin using a triple quadrupole mass spectrometer. In at least some embodiments, the test sample comprises a pesticide, such as one or more pesticides selected from the group consisting of abamectin, acequinocyl, captan, cyfluthrin, cypermethrin, daminozide, fenhexamid, flunicamide, parallethrin, permethrin, and pyrethrin I. In at least some embodiments, the test sample comprises a mycotoxin, such as ochratoxin A.

The test sample, according to at least some embodiments, may include a botanical test sample, an aqueous sample, a clinical sample, and/or an extract of a marijuana or hemp product (e.g., flowers, concentrates, edibles, topicals, and/or smokables).

In at least some embodiments, the mass spectrometer is configured to detect an MRM transition selected from the group consisting of 890.50/305.10 (abamectin), 402.20/343.10 (acequinocyl), 316.90/263.90 (captan), 451.10/191.00 (cyfluthrin), 433.10/191.00 (cypermethrin), 161/10/143.00 (daminozide), 302.10/97.00 (fenhexamid), 230.10/203.00 (flunicamide), 404.10/239.00 (ochratoxin A), 301.20/133.00 (parallethrin), 408.10/183.00 (permethrin), and 329.20/133.00 (pyrethrin I).

This Summary is not exhaustive of the scope of the present aspects and embodiments. Thus, while certain aspects and embodiments have been presented and/or outlined, it should be understood that the present aspects and embodiments are not limited to the aspects and embodiments in this Summary. Indeed, other aspects and embodiments, which may be similar to and/or different from, the aspects and embodiments presented in this Summary, will be apparent from the description, illustrations and/or claims, which follow.

It should be understood that any aspects and embodiments that are described in this Summary and do not appear in the claims that follow are preserved for later presentation in this application or in one or more continuation patent applications.

It should be understood that any aspects and embodiments that are not described in this Summary and do not appear in the claims that follow are also preserved for later presentation or in one or more continuation patent applications.

### BRIEF DESCRIPTION OF THE DRAWINGS

FIG. 1 is a schematic illustration of an embodiment of a method of detecting a pesticide or mycotoxin.

FIG. 2 is a schematic illustration of an embodiment of a reverse phase chromatographic separation system.

FIG. 3A is a schematic illustration of an embodiment of a reverse phase chromatographic separation column comprising a first mobile phase, a second mobile phase, and oxalic acid.

FIG. 3B is a schematic illustration of an embodiment of a reverse phase chromatographic separation column comprising a first mobile phase, a second mobile phase, oxalic acid, and a test sample.

FIG. 4A is a chromatogram of an acetonitrile (ACN) sample spiked with 100 ppb daminozide. Upper trace, with oxalic acid; lower trace, without oxalic acid.

FIG. 4B is a chromatogram of an ACN sample spiked with 100 ppb daminozide. Upper trace, with oxalic acid; lower trace, without oxalic acid.

FIG. 5 is a chromatogram of an ACN sample spiked with 100 ppb flunicamide. Upper trace, with oxalic acid; lower trace, without oxalic acid.

FIG. 6A is a chromatogram of an ACN sample spiked with 100 ppb cyfluthrin. Upper trace, with oxalic acid; lower trace, without oxalic acid.

FIG. 6B is a chromatogram of an ACN sample spiked with 100 ppb cyfluthrin. Upper trace, with oxalic acid; lower trace, without oxalic acid.

FIG. 7A is a chromatogram of an ACN sample spiked with 100 ppb cypermethrin. Upper trace, with oxalic acid; lower trace, without oxalic acid.

FIG. 7B is a chromatogram of an ACN sample spiked with 100 ppb cypermethrin. Upper trace, with oxalic acid; lower trace, without oxalic acid.

FIG. 8A is a chromatogram of an ACN sample spiked with 100 ppb abamectin. Upper trace, with oxalic acid; lower trace, without oxalic acid.

FIG. 8B is a chromatogram of an ACN sample spiked with 100 ppb abamectin. Upper trace, with oxalic acid; lower trace, without oxalic acid.

FIG. 9 is a chromatogram of an ACN sample spiked with 100 ppb captan. Upper trace, with oxalic acid; lower trace, without oxalic acid.

FIG. 10 is a chromatogram of an ACN sample spiked with 100 ppb ochratoxin A. Upper trace, with oxalic acid; lower trace, without oxalic acid.

FIG. 11A is a chromatogram of an ACN sample spiked with 100 ppb fenhexamid. Upper trace, with oxalic acid; lower trace, without oxalic acid.

FIG. 11B is a chromatogram of an ACN sample spiked with 100 ppb fenhexamid. Upper trace, with oxalic acid; lower trace, without oxalic acid.

FIG. 12 is a chromatogram of an ACN sample spiked with 100 ppb acequinocyl. Upper trace, with oxalic acid; lower trace, without oxalic acid.

FIG. 13 is a chromatogram of an ACN sample spiked with 100 ppb permethrin. Upper trace, with oxalic acid; lower trace, without oxalic acid.

FIG. 14 is a chromatogram of an ACN sample spiked with 100 ppb pyrethrin I. Upper trace, with oxalic acid; lower trace, without oxalic acid.

FIG. 15 is a chromatogram of an ACN sample spiked with 100 ppb parallethrin Upper trace, with oxalic acid; lower trace, without oxalic acid.

FIG. 16 is a chromatogram of a cannabis flower extract sample spiked with 100 ppb acequinocyl. Upper trace, with oxalic acid; lower trace, without oxalic acid.

FIG. 17 is a chromatogram of a cannabis flower extract sample spiked with 100 ppb parallethrin. Upper trace, with oxalic acid; lower trace, without oxalic acid.

FIG. 18 is a chromatogram of a cannabis flower extract sample spiked with 100 ppb abamectin. Upper trace, with oxalic acid; lower trace, without oxalic acid.

### DETAILED DESCRIPTION

The ionization efficiency of certain pesticides and mycotoxins is quite low with the use of the most commonly used additives, making it difficult to measure very low levels of these pesticides and mycotoxins in cannabis, food, water, and other matrices. This is particularly problematic for testing cannabis for pesticides and mycotoxins in US states such as Florida and California and in other countries such as Canada which have rigorous and stringent testing regulations. There is a need for improved systems and methods for detecting and quantifying such pesticides and mycotoxins at lower levels in order to meet these rigorous and stringent testing regulations in different matrices.

This disclosure provides liquid chromatography tandem mass spectrometer (LC-MS/MS) methods and systems for detecting low levels of pesticides and mycotoxins in test samples. In the disclosed methods and systems, oxalic acid is used in small amounts (10 μM to 1000 μM) as an additive to conventional mobile phases containing volatile acids (e.g., formic or acetic acid) and neutral salt (e.g., ammonium formate or ammonium acetate).

The use of small amounts of oxalic acid as an additive improves the signal by a factor of from 1.5 to 9 for a number of pesticides and mycotoxins, including non-polar compounds that are difficult to ionize. This improvement permits detection of these compounds at very low levels, with limits of quantitation far below those currently required in US states such as, but not limited to, Florida and California and in other countries such as, but not limited to, Canada for different cannabis matrices.

The disclosed methods and systems are particularly useful for detection and/or quantification of pesticides and mycotoxins in samples comprising cannabis plant material. Unless otherwise specified in this disclosure, “cannabis” encompasses all varieties of cannabis plants. Cannabis plants include, but are not limited to, cannabis plants containing relatively high levels of tetrahydrocannabinol (THC), such as marijuana; and cannabis plants containing lower levels of THC and higher levels of cannabidiol (CBD), such as hemp. The disclosed systems and methods can be applied to detect pesticides and mycotoxins in a variety of cannabis samples, including marijuana and hemp products such as flowers; concentrates (e.g., oils, tinctures, distillates); edibles such as candy (e.g., gummies, chocolates), cooking oil, baked goods, beverages (e.g., milk, water, wine), ice cream; topicals (e.g., gels, ointments, lotions), botanical samples such as other edible plants and plant products (e.g., herbs, vegetables, fruits, edible flowers, spices, olive oil); other medicinal plants and plant products; other plants and plant products which can be smoked (“smokables,” e.g., tobacco, mint, sage); meats; environmental samples (e.g., water); and clinical samples (e.g., blood serum, urine).

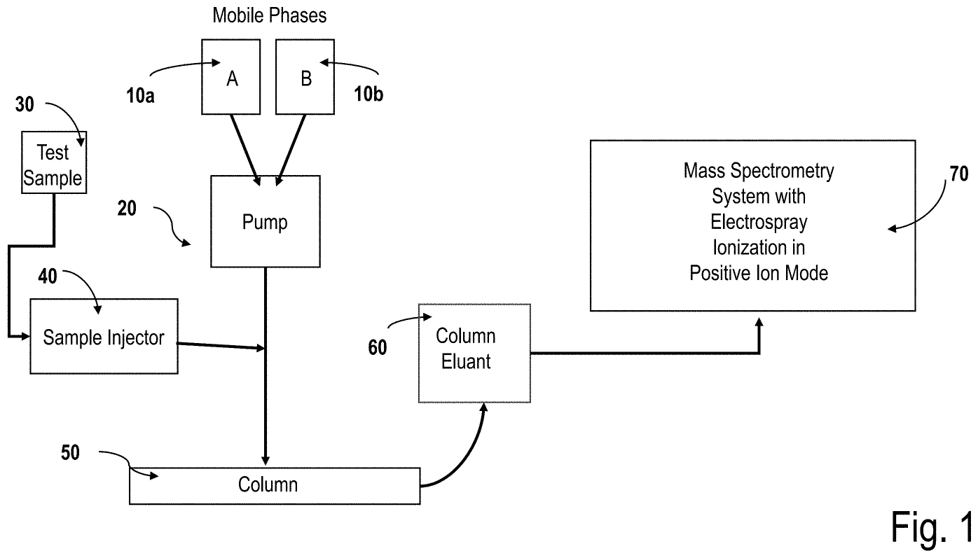

An illustrative and non-limiting embodiment is shown in FIG. 1. In this embodiment, solvent reservoir 10a contains a first mobile phase (A), and solvent reservoir 10b contains a second mobile phase (B).

Any conventional mobile phases can be used. For example, the first mobile phase may comprise water, ammonium formate, and formic acid, and the second mobile phase may comprise methanol, ammonium formate, and formic acid. In the working examples below, each mobile phase comprises 2 mM ammonium formate and 0.1% formic acid; however, the invention is not limited to this gradient, and any LC gradient containing different solvent mobile phases (e.g., water, acetonitrile, methanol and others) and/or different additives (e.g., ammonium acetate, acetic acid and others) can be used.

Each mobile phase independently comprises from 0 μM to 1000 μM oxalic acid. In the Examples below, the concentration of oxalic acid is 83 μM in the first mobile phase and 166 μM in the second mobile phase, but the invention is not limited to these relative or absolute concentrations. The concentration of oxalic acid can vary independently in the two mobile phases from 0 μM to 1000 μM, as long as one of the mobile phases contains a concentration of oxalic acid greater than 0 μM. In some embodiments, the concentration of oxalic acid in the two mobile phases independently varies between 50 μM and 500 μM. In some embodiments, the concentration of oxalic acid in one of the mobile phases is 0 μM, and the concentration of oxalic acid in the other mobile phase varies from 10 μM to 1000 μM (e.g., 50 μM to 500 μM, 10 μM to 500 μM, 50 μM to 750 μM, etc.).

Mobile phases A and B are pumped via pump 20 to a reverse phase chromatographic separation column 50. Any type of reverse phase chromatographic column can be used (e.g., columns in which the solid phase is C18, biphenyl, C4, C8, phenyl, Cl, pentafluorophenyl, C30, polar embedded, polar end-capped, porous graphitic carbon, phenyl-hexyl, alkyl).

Test sample 30 is loaded onto the reverse phase chromatographic separation column via sample injector 40. At this point, the reverse phase chromatographic separation column 50 contains a “mobile phase composition,” which comprises the first mobile phase A, the second mobile phase B, and the test sample. The proportion of the two mobile phases is shifted over time to create a gradient that passes over the column. The resulting column eluant 60 is subjected to an electrospray ionization source in positive ion mode and Multiple Reaction Monitoring (MRM) transitions for pesticide(s) and/or mycotoxin(s) using a tandem MS/MS mass spectrometer system 70.

Reverse Phase Chromatographic Separation System

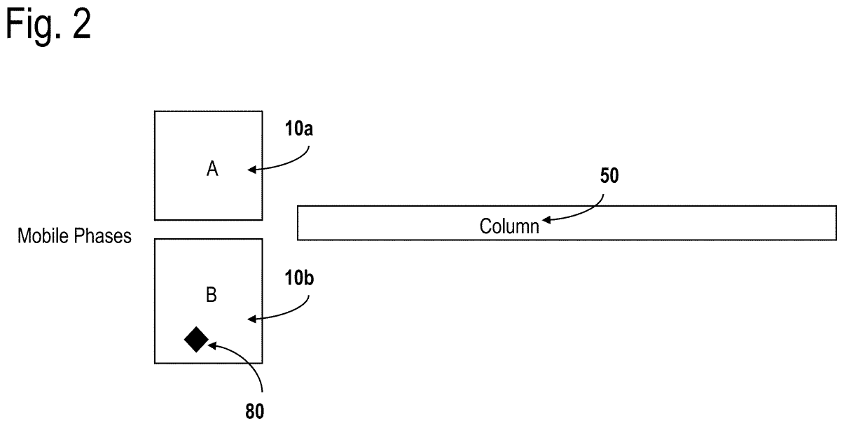

In some embodiments, such as the embodiment shown in FIG. 2, this disclosure provides a reverse phase chromatographic separation system, comprising a reverse phase chromatographic separation column 50, a first mobile phase 10a, and a second mobile phase 10b comprising oxalic acid 80. In other embodiments, each mobile phase comprises oxalic acid. In some embodiments, the first mobile phase comprises water, ammonium formate, and formic acid. In some embodiments, the second mobile phase comprises methanol, ammonium formate, and formic acid.

In some embodiments, the first mobile phase and the second mobile phase each comprise 2 mM ammonium formate and 0.1% formic acid. In some of these embodiments, the first mobile phase comprises water, and the second mobile phase comprises methanol.

In any of the embodiments described above, the first mobile phase comprises a first concentration of oxalic acid of from 0 μM to 1000 μM, and the second mobile phase independently comprises a second concentration of oxalic acid of from 0 μM to 1000 μM, as long as one of the mobile phases comprises a concentration of oxalic acid greater than 0 μM. In some embodiments, the concentration of oxalic acid in the two mobile phases independently varies between 50 μM and 500 μM. In some embodiments, the concentration of oxalic acid in one of the mobile phases is 0 μM, and the concentration of oxalic acid in the other mobile phase varies from 10 μM to 1000 μM (e.g., 50 μM to 500 μM, 10 μM to 500 μM, 50 μM to 750 μM, etc.). In some embodiments, the first mobile phase comprises 83 μM oxalic acid. In some embodiments, the second mobile phase comprises 166 μM oxalic acid.

Reverse Phase Chromatographic Column

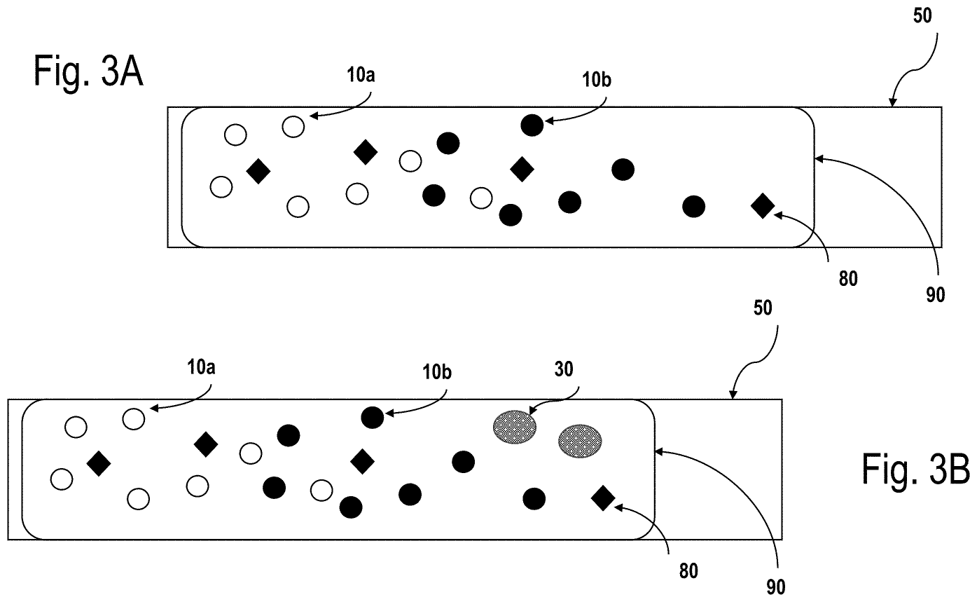

In some embodiments, such as the embodiment shown in FIG. 3A, this disclosure provides a reverse phase chromatographic column comprising a mobile phase composition 90, wherein the mobile phase composition comprises a first mobile phase 10a, a second mobile phase 10b, and oxalic acid 80. In some embodiments, the first mobile phase comprises water, ammonium formate, and formic acid. In some embodiments, the second mobile phase comprises methanol, ammonium formate, and formic acid.

In some embodiments, the first mobile phase and the second mobile phase each comprise 2 mM ammonium formate and 0.1% formic acid. In some of these embodiments, the first mobile phase comprises water, and the second mobile phase comprises methanol.

In any of the embodiments described above, the first mobile phase comprises a first concentration of oxalic acid of from 0 μM to 1000 μM, and the second mobile phase independently comprises a second concentration of oxalic acid of from 0 μM to 1000 μM as long as one of the mobile phases comprises a concentration of oxalic acid greater than 0 μM. In some embodiments, the concentration of oxalic acid in the two mobile phases independently varies between 50 μM and 500 μM. In some embodiments, the concentration of oxalic acid in one of the mobile phases is 0 μM, and the concentration of oxalic acid in the other mobile phase varies from 10 μM to 1000 μM (e.g., 50 μM to 500 μM, 10 μM to 500 μM, 50 μM to 750 μM, etc.). In some embodiments, the first mobile phase comprises 83 μM oxalic acid. In some embodiments, the second mobile phase comprises 166 μM oxalic acid.

In some embodiments the mobile phase composition comprises a test sample 30 (FIG. 3B). In some embodiments, the test sample is a botanical test sample, an environmental sample, or a clinical sample. In some embodiments, the test sample is an extract of a marijuana or hemp product. In some embodiments, the marijuana or hemp product is selected from the group consisting of flowers, concentrates, edibles, topicals, and smokables.

In some embodiments, the test sample comprises a pesticide, e.g., abamectin, acequinocyl, captan, cyfluthrin, cypermethrin, daminozide, fenhexamid, flunicamide, parallethrin, permethrin, pyrethrin I. In some embodiments, the test sample comprises a mycotoxin, e.g., ochratoxin A. The method disclosed here is not limited to above examples of pesticides and mycotoxins and can be extended to other types of pesticides, mycotoxins, and other analytes.

Liquid Chromatography-Tandem Mass Spectrometer System

In some embodiments, the reverse phase chromatographic separation systems or reverse phase chromatographic separation columns described above are part of a liquid chromatography tandem mass spectrometer (LC-MS/MS) system, which also comprises a triple quadrupole mass spectrometer. In some embodiments the triple quadrupole mass spectrometer is a PerkinElmer QSIGHT™ LC/MS/MS mass spectrometer. In other embodiments, any other commercially available liquid chromatography column coupled to mass spectrometer with electrospray ion source can be used to demonstrate signal improvement for different pesticides, mycotoxins and other compounds.

The spectrometer can be configured to detect MRM transitions associated with various pesticides and mycotoxins, such as 890.50/305.10 (abamectin), 402.20/343.10 (acequinocyl), 316.90/263.90 (captan), 451.10/191.00 (cyfluthrin), 433.10/191.00 (cypermethrin), 161/10/143.00 (daminozide), 302.10/97.00 (fenhexamid), 230.10/203.00 (flunicamide), 404.10/239.00 (ochratoxin A), 301.20/133.00 (parallethrin), 408.10/183.00 (permethrin), and 329.20/133.00 (pyrethrin I).

Methods for Detecting Pesticides and Mycotoxins

This disclosure provides methods for detecting a pesticide and/or a mycotoxin in a test sample. A test sample is processed using a reverse phase chromatographic separation column as described above, which provides a liquid chromatography (LC) column eluant. The LC column eluant is then tested for the presence of the pesticide and/or mycotoxin using a triple quadrupole mass spectrometer as described above, configured to detect one or more MRM transitions associated with particular pesticides and/or mycotoxins. In some embodiments, the MRM transitions are selected from the group consisting of 890.50/305.10 (abamectin), 402.20/343.10 (acequinocyl), 316.90/263.90 (captan), 451.10/191.00 (cyfluthrin), 433.10/191.00 (cypermethrin), 161/10/143.00 (daminozide), 302.10/97.00 (fenhexamid), 230.10/203.00 (flunicamide), 404.10/239.00 (ochratoxin A), 301.20/133.00 (parallethrin), 408.10/183.00 (permethrin), and 329.20/133.00 (pyrethrin I).

Those skilled in the art will appreciate that there are numerous variations and permutations of the above described embodiments that fall within the scope of the appended claims.

### EXAMPLE 1. Improved Detection of Pesticides and Mycotoxins in Acetonitrile

This example demonstrates the improved detection sensitivity of pesticides and mycotoxins spiked into acetonitrile using an ESI source method in positive ion mode.

Test samples were prepared by spiking the various compounds tested below in acetonitrile.

Chromatographic separation was conducted using a reverse phase PerkinElmer Quasar SPP Pesticides (4.6×100 mm, 2.7 μm) C18 LC column, and detection was achieved using a PerkinElmer QSIGHT™ LC/MS/MS mass spectrometer with electrospray ion source. The composition of mobile phase A was water, 2 mM ammonium formate, 0.1% formic acid, and 0.00075% (83 μM) oxalic acid. The composition of mobile phase B was methanol, 2 mM ammonium formate, 0.1% formic acid, and 0.0015% (166 μM) oxalic acid. The LC flow rate was 0.8 ml/min, and the injection volume was 3 μL. The LC gradient parameter was 5% mobile phase B to 100% mobile phase B in 18 minutes, including equilibration time.

Parameters for the mass spectrometer were as follows. The ionization source was ESI in positive ion mode. Source temperature was 290° C., the hot surface induced desolvation (HSID™) temperature interface was 200° C. Nebulizing gas (air) was 350 arbitrary units, and drying gas (nitrogen) was 150 arbitrary units.

All instrument control, data acquisition and data processing was performed using the Simplicity 3Q™ software platform.

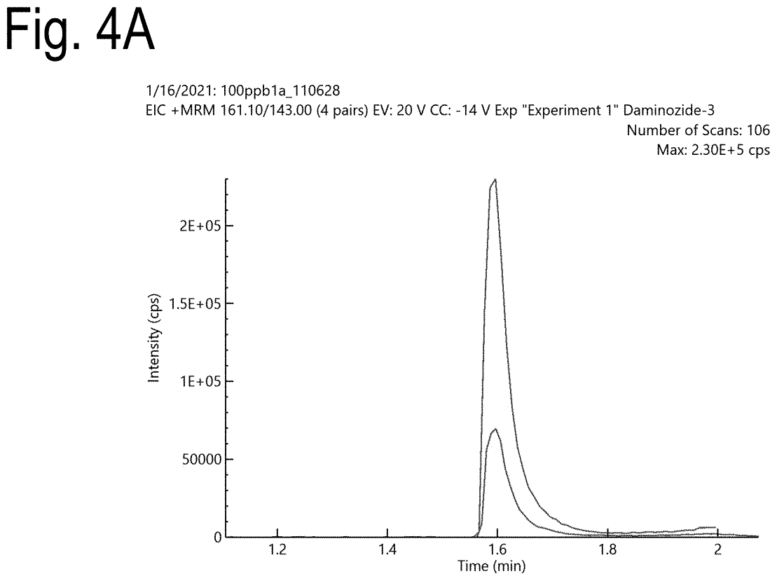

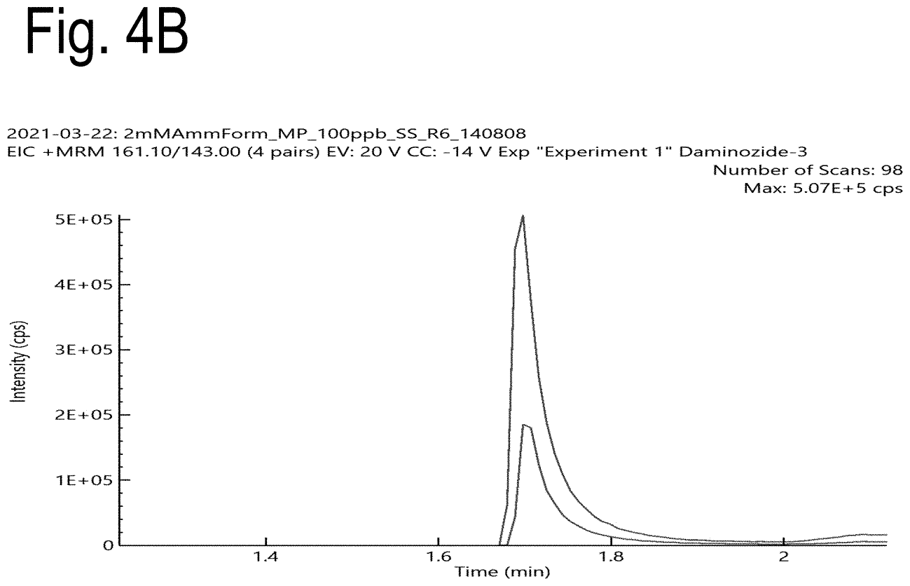

The results are shown in FIGS. 4-15, which compare the signal obtained with the addition of oxalic acid (upper trace) and without the addition of oxalic acid (lower trace). In each case, addition of oxalic acid resulted in at least a 1.5-fold improvement in signal detection.

FIGS. 4A and 4B show a 2.5-fold and a 3.3-fold improvement, respectively, in signal detection for daminozide.

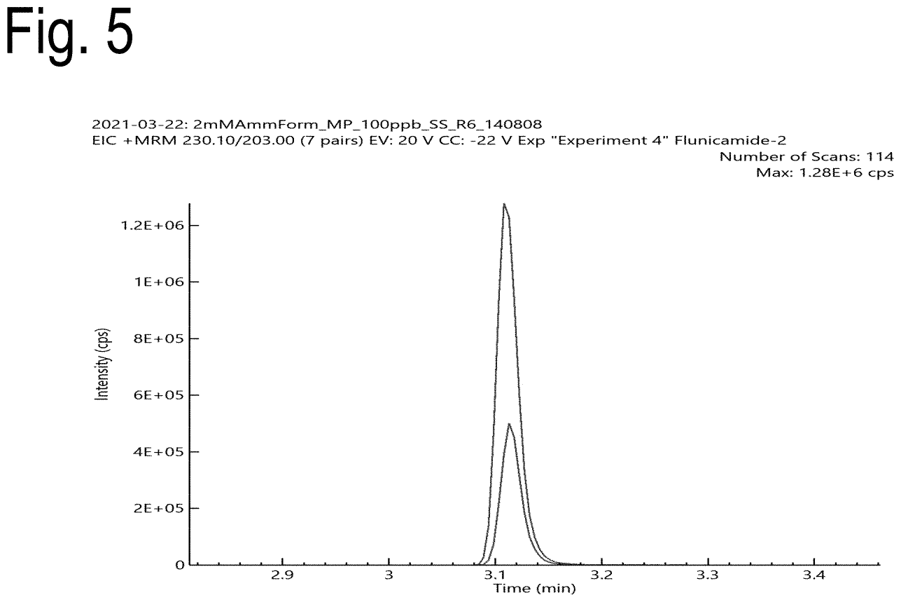

FIG. 5 shows a five-fold improvement in signal detection for flunicamide.

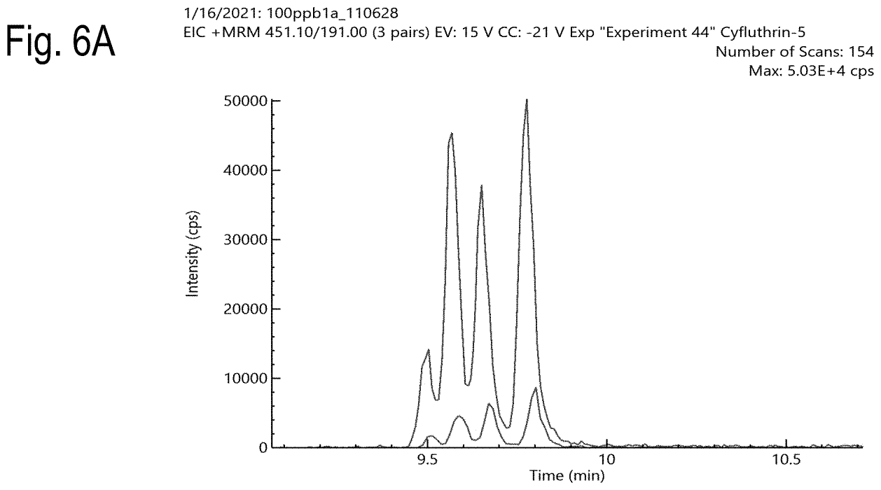

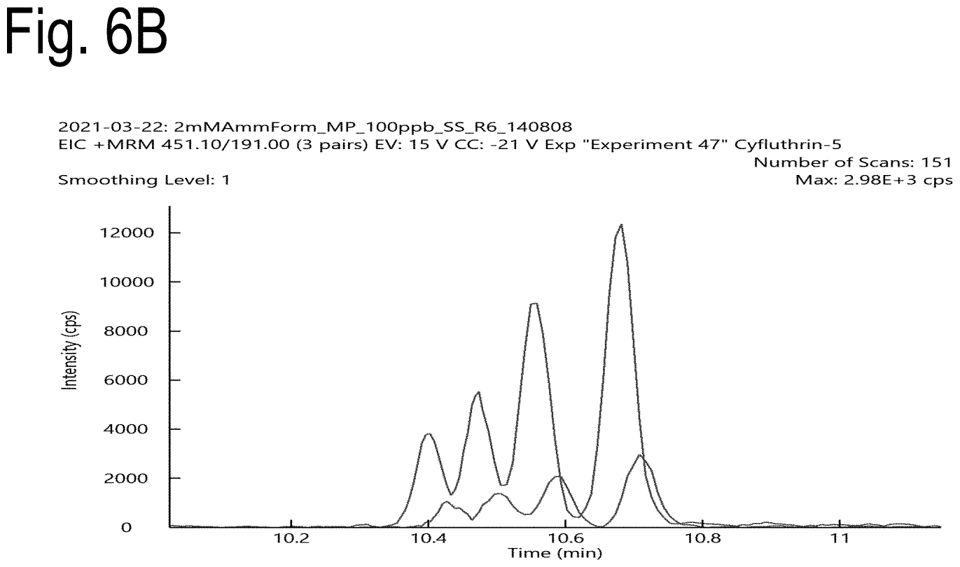

FIGS. 6A and 6B show a 3.5-fold and four-fold improvement in signal detection for cyfluthrin, respectively, over four isomers.

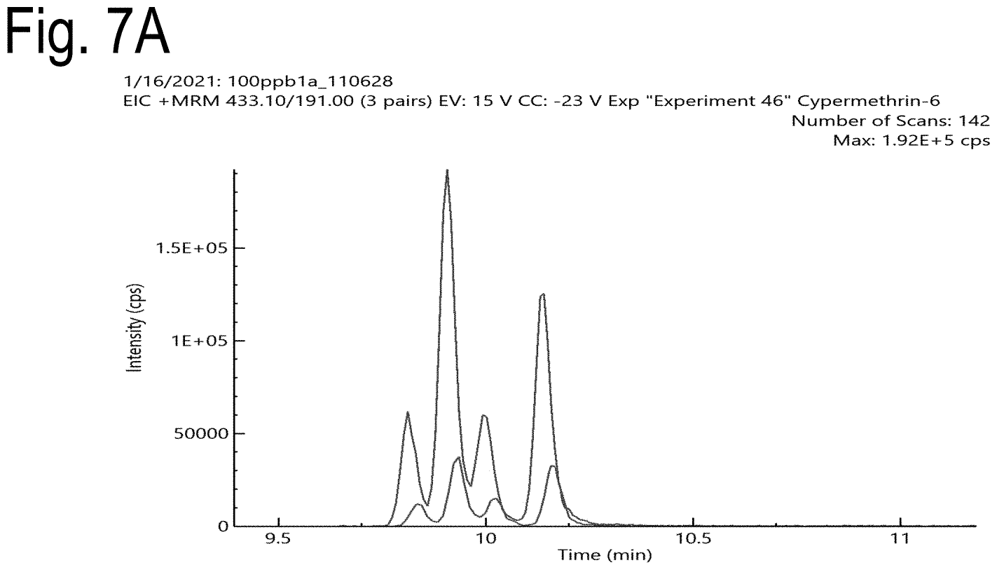

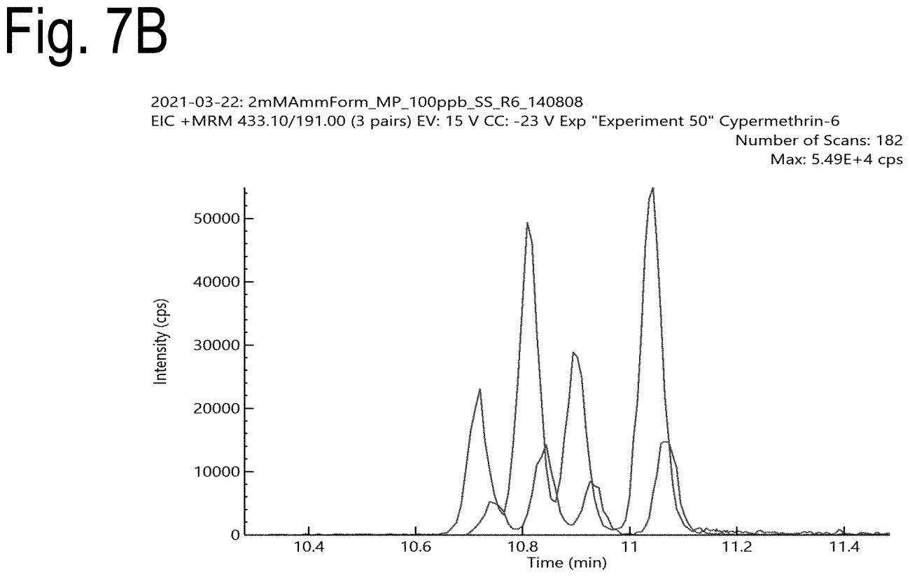

FIGS. 7A and 7B show a five-fold improvement in signal detection for cypermethrin, over four isomers.

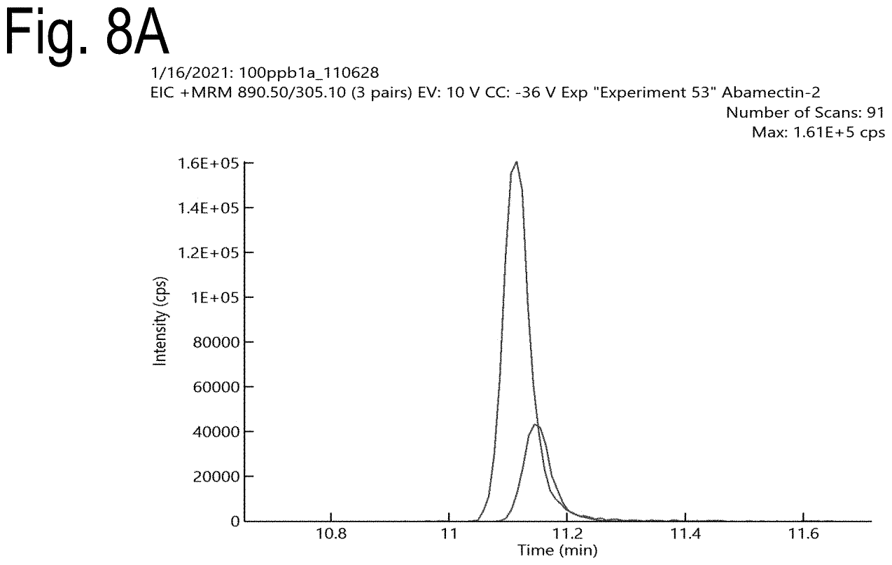

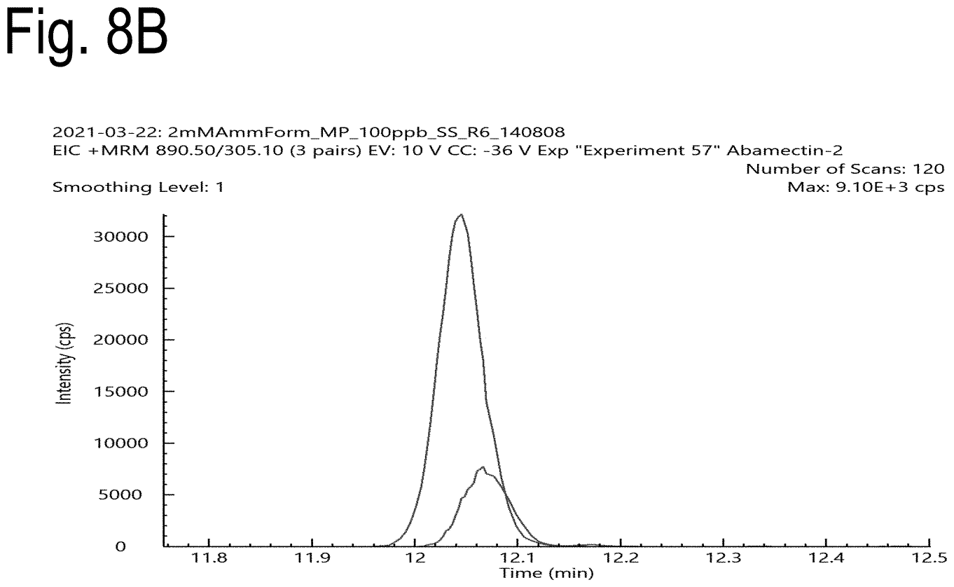

FIGS. 8A and 8B show a four-fold improvement in signal detection for abamectin.

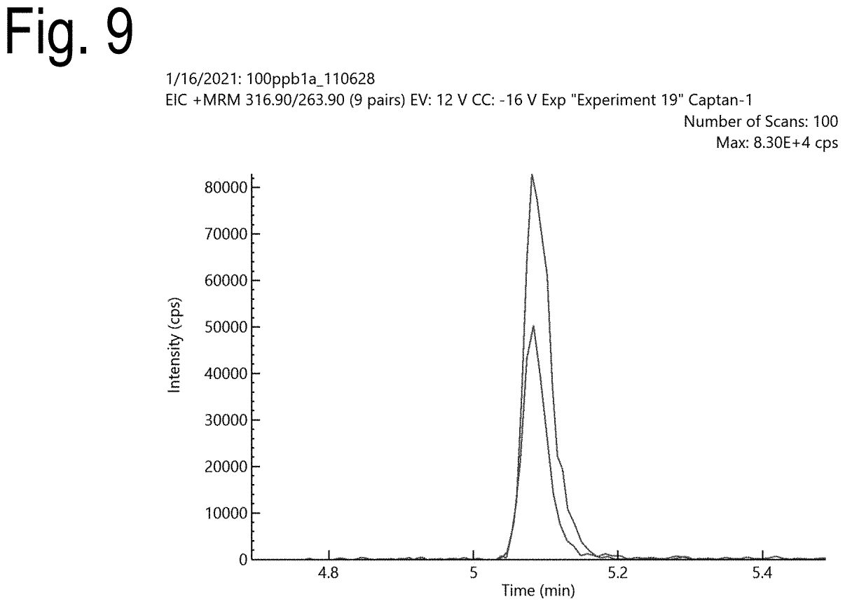

FIG. 9 shows a 1.5-fold improvement in signal detection for captan.

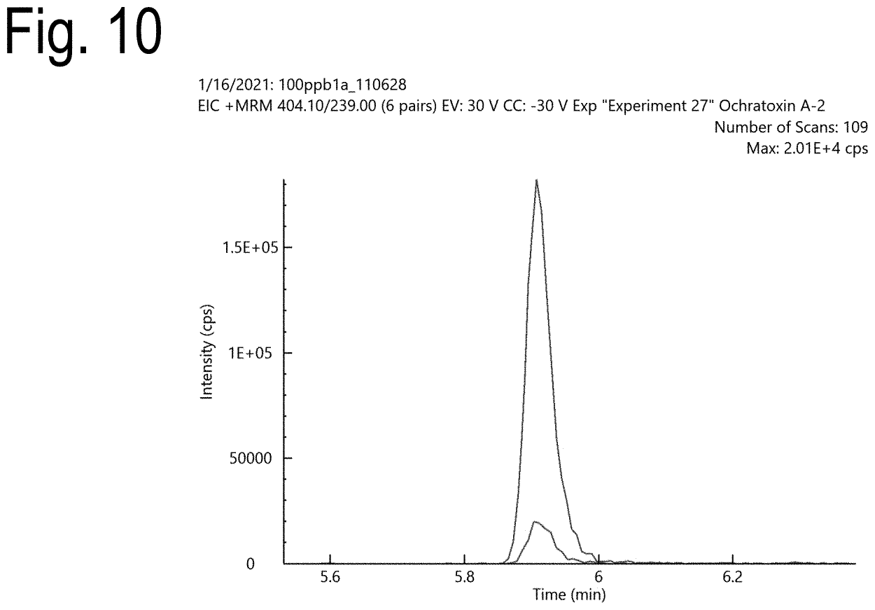

FIG. 10 shows a nine-fold improvement in signal detection for ochratoxin A.

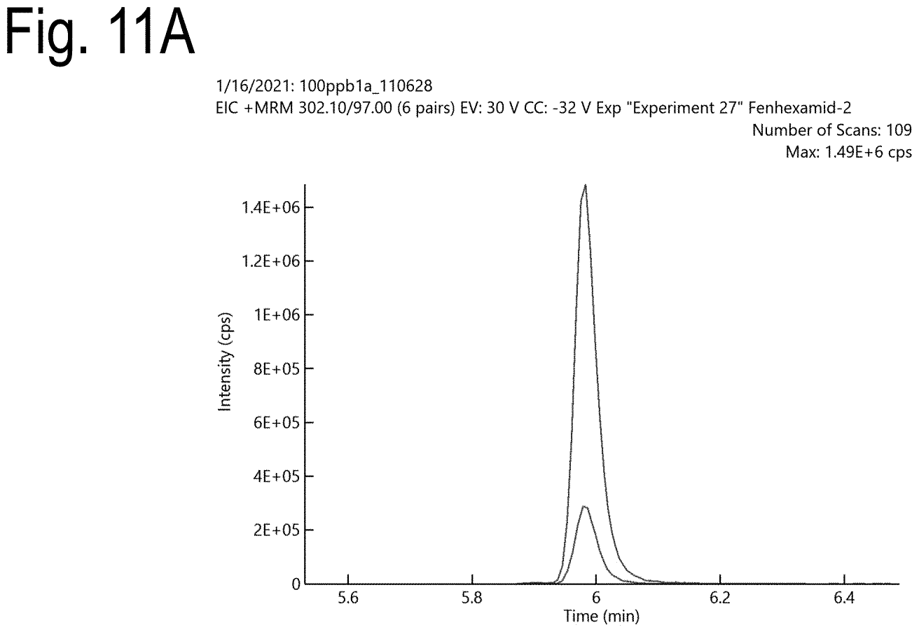

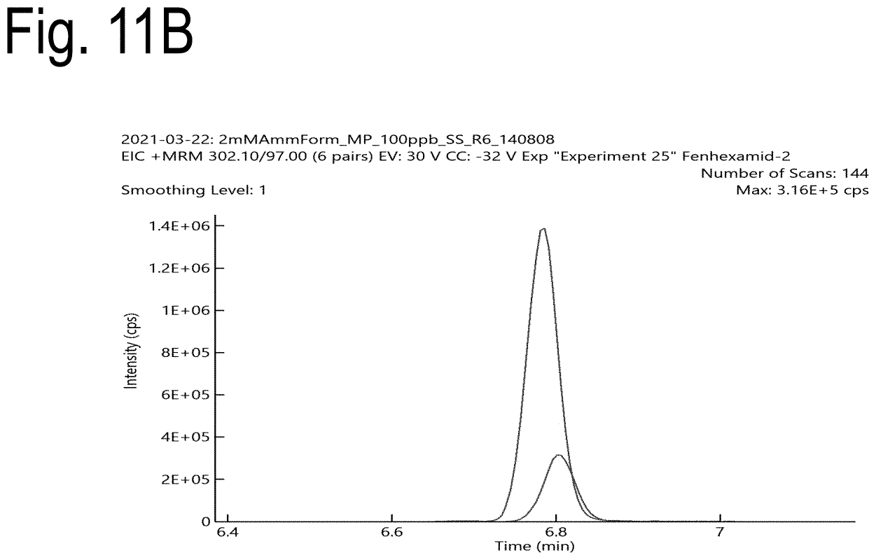

FIGS. 11A and 11B show a five-fold improvement in signal detection for fenhexamid.

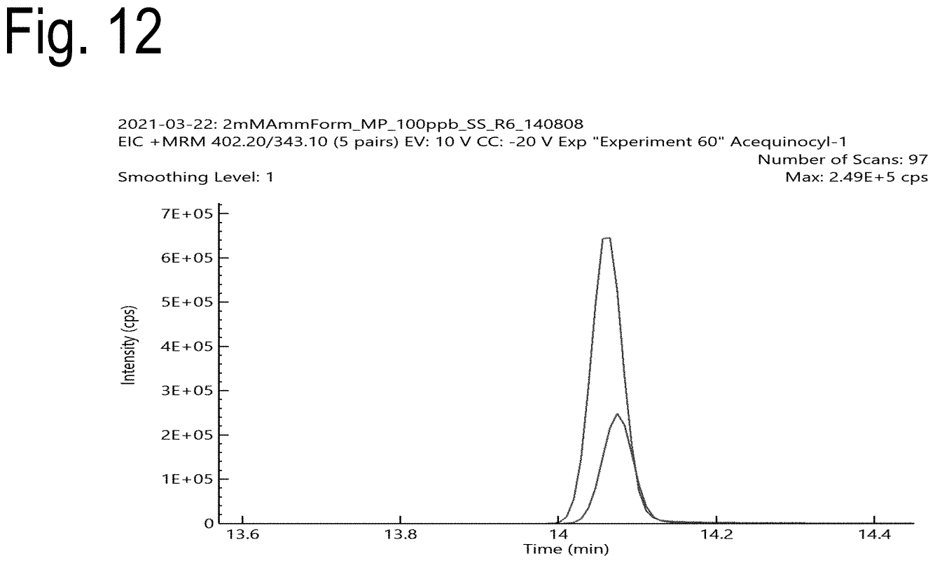

FIG. 12 shows a three-fold improvement in signal detection for acequinocyl.

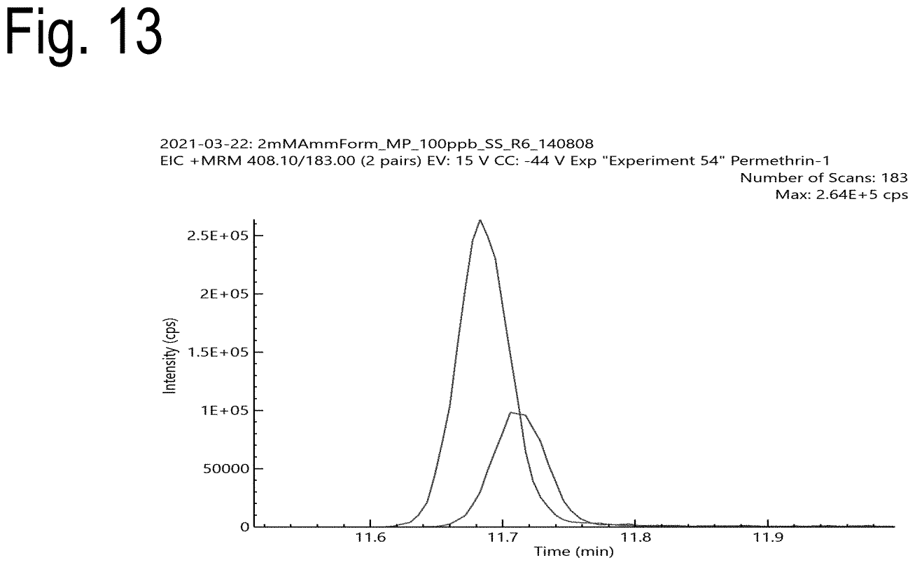

FIG. 13 shows a 2.5-fold improvement in signal detection for permethrin.

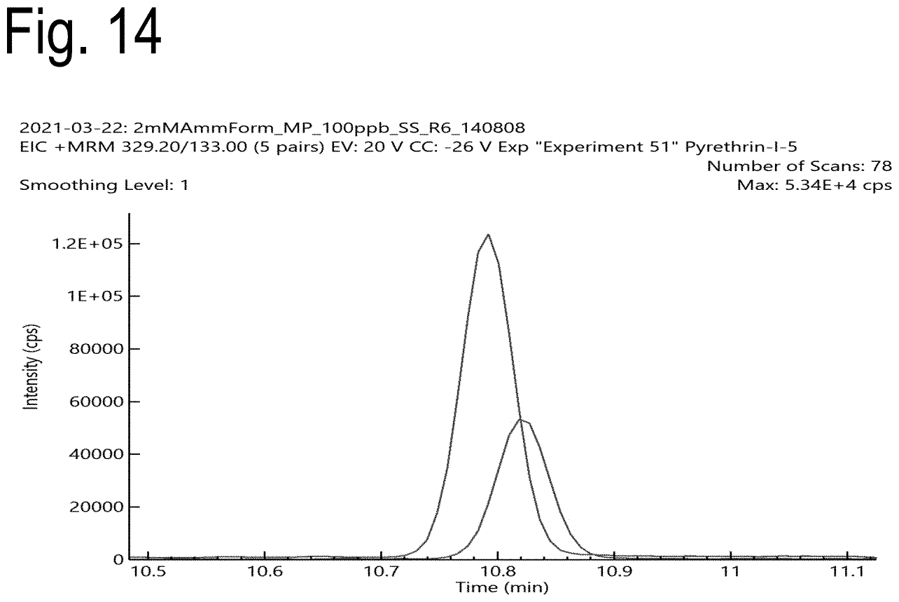

FIG. 14 shows a 2.3-fold improvement in signal detection for pyrethrin I.

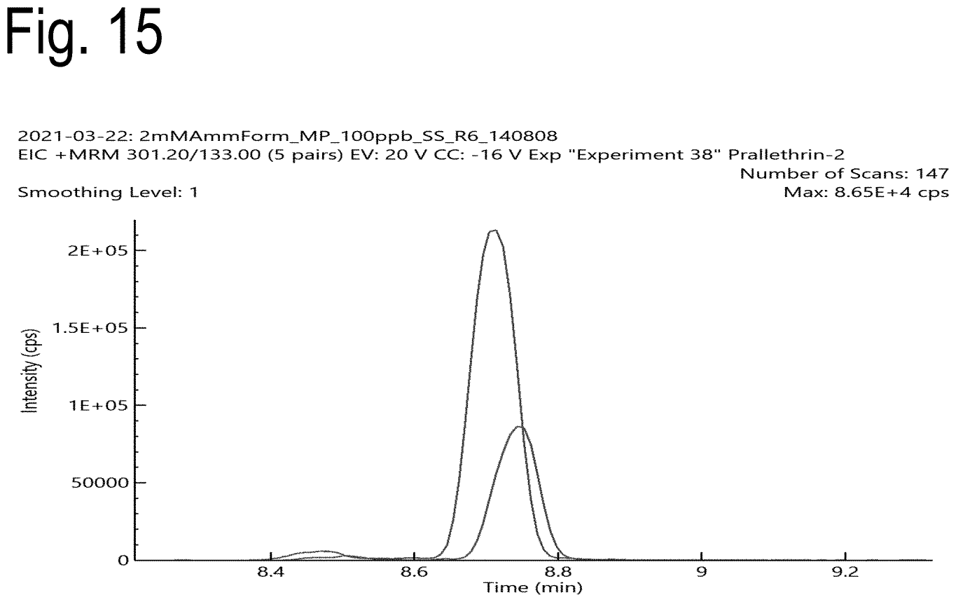

FIG. 15 shows a 2.3-fold improvement in signal detection for parallethrin.

### EXAMPLE 2. Improved Detection of Pesticides in Cannabis Flower Extracts

This example demonstrates the improved detection sensitivity of pesticides in cannabis flower extracts using an ESI source method in positive ion mode. Cannabis flower samples were prepared as follows, with 10-fold dilution. Approximately 5 grams of cannabis flower were ground finely. One gram was placed in a 50 mL centrifuge tube. Five mL of LC/MS grade acetonitrile were added to the tube. The tube was capped, placed on a multi-tube vortex mixer, vortexed for 10 minutes, and centrifuged for 10 minutes at 3000 rpm. The solvent was filtered into a 5 mL glass amber vial using 0.22 micron nylon syringe-filter, and the vial was capped and labeled.

A 0.5 mL aliquot of the extracted sample was placed into a 2 mL HPLC vial, diluted with 0.49 mL of LC/MS grade acetonitrile, mixed, and spiked with 10 μL of acequinocyl, parallethrin, or abamectin to a concentration of 100 ppb.

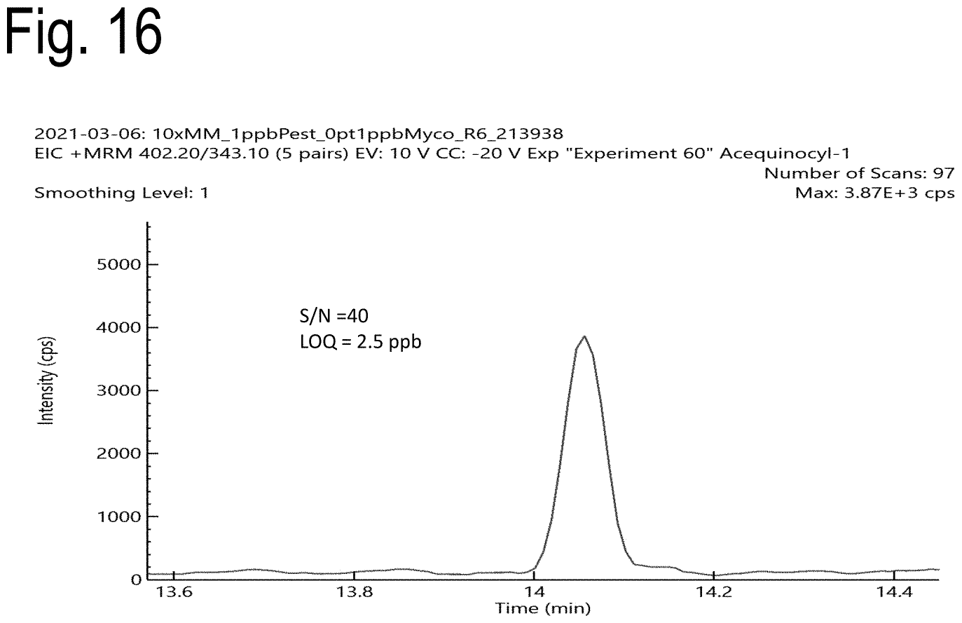

LC-MS/MS method parameters were as described in Example 1. The results are shown in FIGS. 16-18.

For acequinocyl, addition of the oxalic acid resulted in a signal to noise (S/N) ratio for acequinocyl of 40 and a limit of quantitation (LOQ) of 2.5 ppb (FIG. 16), well below the current LOQ of 50 ppb required in Florida for this compound in cannabis flower.

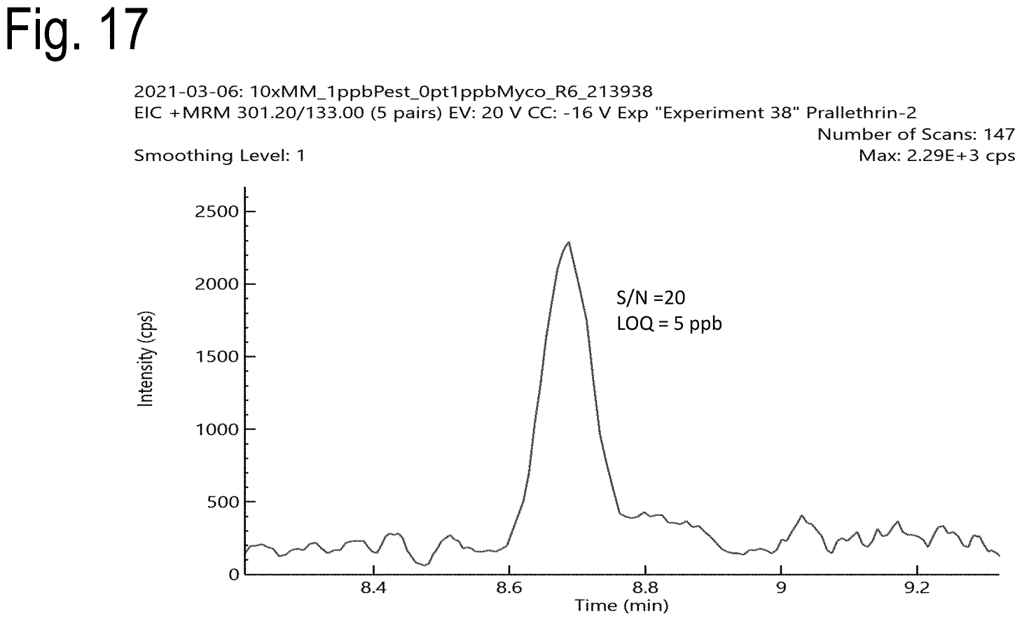

For parallethrin, use of the oxalic acid additive resulted in an S/N of 20 and an LOQ of 5 ppb for parallethrin (FIG. 17), well below the current LOQ of 50 ppb required in Florida for this compound in cannabis.

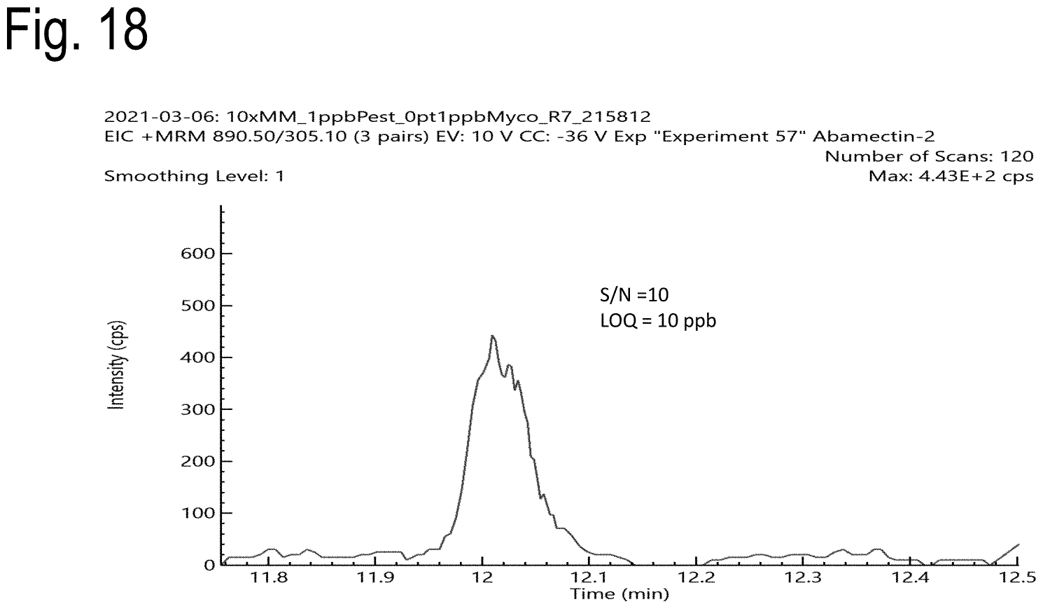

For abamectin, use of the oxalic acid additive resulted in a S/N of 10 and an LOQ of 10 ppb (FIG. 18), well below the current LOQ of 50 ppb required in Florida for this compound in cannabis flower.

The present technology has been described herein with reference to the accompanying drawings, in which illustrative embodiments of the technology are shown. In the drawings, the relative sizes of regions or features may be exaggerated for clarity or shown schematically. As may be recognized by those of ordinary skill in the pertinent art based on the teachings herein, numerous changes may be made to the above-described and other embodiments of the present invention without departing from the scope of the invention. For example, the method, system, and/or column may be used to detect low levels of pesticides and/or mycotoxins in various matrices, such as, but not limited to, food or water. In addition, the devices, systems, and/or methods may include fewer or more components or features than the embodiments described herein. Thus, this technology may be embodied in many different forms and should not be construed as limited to the embodiments set forth herein; rather, these embodiments are provided so that this disclosure will be thorough and complete, and will fully convey the scope of the technology to those skilled in the art.

It will be understood that, although the terms first, second, etc. may be used herein to describe various elements, components, concentrations, and/or phases, these elements, components, concentrations, and/or phases should not be limited by these terms. These terms are only used to distinguish one element, component, concentration, or phase from another element, component, concentration, or phase. Thus, a first element, component, concentration, or phase discussed below could be termed a second element, component, concentration, or phase without departing from the teachings of the present technology.

As used herein, the singular forms “a,” “an,” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items.

Many alterations and modifications may be made by those having ordinary skill in the art, given the benefit of present disclosure, without departing from the spirit and scope of the invention. Therefore, it must be understood that the illustrated embodiments have been set forth only for the purposes of example, and that it should not be taken as limiting the invention as defined by the following claims. The following claims, therefore, are to be read to include not only the combination of elements which are literally set forth, but all equivalent elements for performing substantially the same function in substantially the same way to obtain substantially the same result. The claims are thus to be understood to include what is specifically illustrated and described above, what is conceptually equivalent, and also what incorporates the essential idea of the invention.

1. A reverse phase chromatographic separation column comprising a mobile phase composition, wherein the mobile phase composition comprises a first mobile phase, a second mobile phase, and oxalic acid.

2. The reverse phase chromatographic separation column of claim 1, wherein the first mobile phase comprises water, ammonium formate, and formic acid.

3. The reverse phase chromatographic separation column of claim 1, wherein the second mobile phase comprises methanol, ammonium formate, and formic acid.

4. The reverse phase chromatographic separation column of claim 1, wherein the first mobile phase comprises a first concentration of oxalic acid of from 10 μM to 1000 μM.

5. The reverse phase chromatographic separation column of claim 1, wherein the first mobile phase and the second mobile phase independently comprise a concentration of oxalic acid of from 0 μM to 1000 μM, provided that at least one of the first and second mobile phases comprises a concentration of oxalic acid greater than 0 μM.

6. The reverse phase chromatographic separation column of claim 5, wherein the first and second concentrations of oxalic acid independently are from 50 μM to 500 μM.

7. The reverse phase chromatographic separation column of claim 1, wherein the first mobile phase comprises 83 μM oxalic acid.

8. The reverse phase chromatographic separation column of claim 1, wherein the second mobile phase comprises 166 μM oxalic acid.

9. The reverse phase chromatographic separation column of claim 1, wherein the first mobile phase and the second mobile phase each comprise 2 mM ammonium formate and 0.1% formic acid.

10. The reverse phase chromatographic separation column of claim 1, wherein the mobile phase composition comprises a test sample.

11. The reverse phase chromatographic separation column of claim 10, wherein the test sample is a botanical test sample, an environmental sample, or a clinical sample.

12. The reverse phase chromatographic separation column of claim 10, wherein the test sample is an extract of a marijuana or hemp product.

13. The reverse phase chromatographic separation column of 12, wherein the marijuana or hemp product is selected from the group consisting of flowers, concentrates, edibles, topicals, and smokables.

14. The reverse phase chromatographic separation column of claim 10, wherein the test sample comprises a pesticide.

15. The reverse phase chromatographic separation column of claim 14, wherein the pesticide is selected from the group consisting of abamectin, acequinocyl, captan, cyfluthrin, cypermethrin, daminozide, fenhexamid, flunicamide, parallethrin, permethrin, and pyrethrin I.

16. The reverse phase chromatographic separation column of claim 10, wherein the test sample comprises a mycotoxin.

17. The reverse phase chromatographic separation column of claim 16, wherein the mycotoxin is ochratoxin A.

18. A reverse phase chromatographic separation system, comprising a reverse phase chromatographic separation column, a first mobile phase comprising oxalic acid, and a second mobile phase comprising oxalic acid.

19. The reverse phase chromatographic separation system of claim 18, wherein the first mobile phase comprises water, ammonium formate, and formic acid.

20. The reverse phase chromatographic separation system of claim 18, wherein the second mobile phase comprises methanol, ammonium formate, and formic acid.

21. The reverse phase chromatographic separation system of claim 18, wherein the first mobile phase comprises a first concentration of oxalic acid.

22. The reverse phase chromatographic separation system of claim 18, wherein the second mobile phase comprises a second concentration of oxalic acid.

23. The reverse phase chromatographic separation system of claim 18, wherein the first and second concentrations of oxalic acid independently are from 0 μM to 1000 μM, provided that at least one of the first and second mobile phases comprises a concentration of oxalic acid greater than 0 μM.

24. The reverse phase chromatographic separation system of claim 18, wherein the first mobile phase comprises 83 μM oxalic acid.

25. The reverse phase chromatographic separation system of claim 18, wherein the second mobile phase comprises 166 μM oxalic acid.

26. The reverse phase chromatographic separation system of claim 18, wherein the first mobile phase and the second mobile phase each comprise 2 mM ammonium formate and 0.1% formic acid.

27. A liquid chromatography tandem mass spectrometer (LC-MS/MS) system, comprising:
(a) a reverse phase chromatographic separation column comprising a mobile phase composition, wherein the mobile phase composition comprises a first mobile phase, a second mobile phase, and oxalic acid, or a reverse phase chromatographic separation system comprising a reverse phase chromatographic separation column, a first mobile phase comprising oxalic acid, and a second mobile phase comprising oxalic acid; and
(b) a triple quadrupole mass spectrometer.

28. The LC-MS/MS system of claim 27, wherein the triple quadrupole mass spectrometer is configured to detect an MRM transition selected from the group consisting of 890.50/305.10 (abamectin), 402.20/343.10 (acequinocyl), 316.90/263.90 (captan), 451.10/191.00 (cyfluthrin), 433.10/191.00 (cypermethrin), 161/10/143.00 (daminozide), 302.10/97.00 (fenhexamid), 230.10/203.00 (flunicamide), 404.10/239.00 (ochratoxin A), 301.20/133.00 (parallethrin), 408.10/183.00 (permethrin), and 329.20/133.00 (pyrethrin I).

29. A method of detecting a pesticide or a mycotoxin in a test sample, comprising:
(a) processing a test sample using a reverse phase chromatographic separation column comprising a mobile phase composition, wherein the mobile phase composition comprises a first mobile phase, a second mobile phase, and oxalic acid, or a reverse phase chromatographic separation system comprising a reverse phase chromatographic separation column, a first mobile phase comprising oxalic acid, and a second mobile phase comprising oxalic acid to provide a liquid chromatography (LC) column eluant; and
(b) analyzing the LC column eluant for the presence of the pesticide or the mycotoxin using a triple quadrupole mass spectrometer.

30. The method of claim 29, wherein the test sample comprises a pesticide.

31. The method of claim 30, wherein the pesticide is selected from the group consisting of abamectin, acequinocyl, captan, cyfluthrin, cypermethrin, daminozide, fenhexamid, flunicamide, parallethrin, permethrin, and pyrethrin I.

32. The method of claim 29, wherein the test sample comprises a mycotoxin.

33. The method of claim 32, wherein the mycotoxin is ochratoxin A.

34. The method of claim 29, wherein the test sample is a botanical test sample, an aqueous sample, or a clinical sample.

35. The method of claim 29, wherein the test sample is an extract of a marijuana or hemp product.

36. The method of claim 35, wherein the marijuana or hemp product is selected from the group consisting of flowers, concentrates, edibles, topicals, and smokables.

37. The method of claim 29, wherein mass spectrometer is configured to detect an MRM transition selected from the group consisting of 890.50/305.10 (abamectin), 402.20/343.10 (acequinocyl), 316.90/263.90 (captan), 451.10/191.00 (cyfluthrin), 433.10/191.00 (cypermethrin), 161/10/143.00 (daminozide), 302.10/97.00 (fenhexamid), 230.10/203.00 (flunicamide), 404.10/239.00 (ochratoxin A), 301.20/133.00 (parallethrin), 408.10/183.00 (permethrin), and 329.20/133.00 (pyrethrin I).
